(19) United States
(12) Patent Application Publication
(10) Pub. No.: US 2023/0414728 A1
(43) Pub. Date: Dec. 28, 2023

(54) METHODS OF TREATING SKIN CANCERS

(71) Applicant: MEDIWOUND LTD., Yavne (IL)

(72) Inventors: Lior ROSENBERG, Omer (IL); Dafna GEBLINGER, Rehovot (IL); Eilon ASCULAI, Lehavim (IL)

(73) Assignee: MEDIWOUND LTD., Yavne (IL)

(21) Appl. No.: 18/249,097

(22) PCT Filed: Oct. 18, 2021
(86) PCT No.: PCT/IL2021/051227

§ 371 Date: Apr. 14, 2023

Related U.S. Application Data
(60) Provisional application No. 63/093,312, filed on Oct. 19, 2020.

(51) Int. Cl. A61K 38/48; A61K 9/00; A61K 9/06; A61K 47/36; A61K 47/26; A61K 47/02; A61K 47/10; A61P 35/00; A61P 17/02

(52) U.S. Cl. CPC A61K 38/4873; A61K 9/0014; A61K 9/06; A61K 47/36; A61K 47/26; A61K 47/02; A61K 47/10; A61P 35/00; A61P 17/02; C12Y 304/22032

(57) ABSTRACT

The present invention provides methods for treating skin cancers utilizing pharmaceutical formulations in the form of a hydrogel that includes a proteolytic enzyme mixture obtained from bromelain and a water-soluble gelling agent. In particular, the method is useful for treating non-melanoma skin cancers, pre-cancerous skin lesions, and benign skin lesions.

### FIELD OF THE INVENTION

The present invention relates to methods of treating skin lesions. Particularly, the present invention relates to pharmaceutical formulations in the form of a hydrogel which comprise a proteolytic enzyme mixture obtained from bromelain and a water-soluble gelling agent, for use in treating non-melanoma skin cancers, pre-cancerous skin lesions, and benign skin lesions.

### BACKGROUND OF THE INVENTION

Skin cancer is considered to be the most common form of cancer. There are three main types of skin cancers due to solar radiation: basal-cell carcinoma (also termed basal-cell skin cancer, BCC), squamous-cell carcinoma (also termed squamous-cell skin cancer, SCC) and melanoma. BCC and SCC are known as non-melanoma skin cancers (NMSCs).

Basal cell carcinoma Basal cell carcinoma (BCC) is the most common form of skin cancer and the most common form of cancer of any type in the United States. It develops in the basal germinative cell layer of the epidermis, often on sun-exposed areas of the skin. Although BCC rarely spreads (i.e., metastasizes) to other parts of the body, it can be very destructive and disfiguring. BCC can cause local tissue destruction that may lead to disfigurement or functional impairment of surrounding non-cancerous tissue. Disfigurement may be a particular concern of BCC patients because many BCC tumors occur on the sun-exposed skin of the head and neck. Larger tumors, tumors that have been present for long periods of time, and tumors that have recurred after initial therapy may be biologically more aggressive and especially difficult to cure. While the mortality rate of BCC is relatively low, its increasing incidence and prolonged morbidity result in a disease that is costly to treat. Once a person has developed a BCC, there is a significantly increased risk of developing subsequent BCC at other sites.

A wide variety of surgical and non-surgical therapies are available for BCC. Nonsurgical therapies include radiation therapy, chemotherapy with agents such as 5-fluorouracil (5-FU), and immunotherapy such as with imiquimod. These therapies can be useful for definitive treatment of primary tumors and some recurrent BCC tumors and for relieving symptoms associated with inoperable tumors. However, some of these therapies can also have significant unpleasant side effects. Side effects of radiation therapy and certain chemotherapies are well documented and include hair loss, mouth sores, loss of appetite, nausea and vomiting, diarrhea or constipation, increased risk of infections, easy bruising and bleeding, and fatigue.

Surgical therapies include excision, curettage and electrosurgery, cryosurgery, Mohs micrographic surgery, and laser surgery. Excision is useful for both primary and recurrent tumors and has the advantage of allowing for histological assessment of surgical margins. Curettage and electrosurgery involve alternately removing soft tumor tissue with a curette and then destroying the remaining tissue margins by electrodesiccation, electrocautery, or electrocoagulation. The procedure may be repeated as necessary. Cryosurgery involves freezing the tumor to a temperature that kills the cells of the tumor. The dead tumor cells can be removed by, for example, curettage. Mohs micrographic surgery (MMS) involves repeated surgical removal, layer by layer, freezing and staining each layer, followed by immediate microscopic examination of the layers to determine if there are any remaining cancerous cells, until none are detected. This is followed by closure of the skin defect with sutures, skin grafts, plastic surgery, if necessary, or allowing it to heal by scarring. MMS can increase the likelihood that the entire tumor is removed and minimize the amount of normal tissue that is removed. Laser surgery involves using a laser to vaporize tumor cells. Alternatively, the laser may be used in lieu of a scalpel blade for excisional surgery.

### Squamous Cell Skin Carcinoma

Squamous cell carcinoma of the skin (SCC), also known as cutaneous squamous cell carcinoma (cSCC), is the second most common form of skin cancer. It is characterized by abnormal, accelerated growth of squamous cells which constitute the middle and outer layers of the epidermis.

SCC can appear as scaly red patches, open sores, rough, thickened or wart-like skin, or raised growths with a central depression. SCC may crust over, itch or bleed.

While the majority of SCCs can be successfully treated, these lesions, if allowed to grow, can become disfiguring, dangerous and even deadly. Untreated SCCs can become invasive, grow into deeper layers of skin, and spread to other parts of the body.

Small lesions of SCC are typically treated by curettage and electrodesiccation (electrosurgery), cryosurgery, laser surgery, and photodynamic therapy (PDT). When the SCC lesion is large, excisional surgery, Mohs surgery, and radiation are typically used. When SCC of the skin spreads to other parts of the body, drug treatments are recommended, including chemotherapy with cisplatin and 5-fluorouracil (5-FU), drug targeted therapy with EGFR inhibitors, and immunotherapy with cemiplimab (Libtayo) or pembrolizumab (Keytruda).

### Bromelain

Bromelain is an extract derived from the pineapple stem which has been shown to have proteolytic, anti-inflammatory, anti-proliferative and anti-metastatic activities.

The proteolytic properties of bromelain are currently being used for selective removal of dead and necrotic tissues from burns and wounds (debridement) with the preservation of the viable, intact skin.

Numerous in-vitro and in-vivo studies described the anti-cancer activity of bromelain. It was demonstrated that bromelain inhibits nuclear factor-κB (NF-κB) translocation through G2/M arrest to apoptosis in human epidermoid carcinoma and melanoma cells. Bromelain was also shown to selectively induce apoptosis in skin tumor cells by upregulation of p53 expression and initiation of the mitochondrial apoptotic pathway via increased Bax expression and cytochrome C release. In addition, bromelain was shown to decrease the activity of cell survival regulators, such as Akt and extracellular signal-regulated kinases, thus promoting apoptotic cell death in tumors. Bromelain treatment of established mouse tumor cell lines was demonstrated to result in inhibition of cell growth and matrigel invasion (Rathnavelu V, Alitheen N B, Sohila S, Kanagesan S, Ramesh R. Potential role of bromelain in clinical and therapeutic applications. Biomed Rep. 2016; 5(3):283-288. doi:10.3892/br.2016.720).

Administration of enzymes isolated from bromelain for the treatment of diseases and conditions including cancer was previously disclosed. For example, U.S. Pat. No. 5,824,305 to Mynott discloses a method of treating various diseases and conditions which are dependent on cyclic nucleotide pathways with purified stem bromelain protease. According to U.S. Pat. No. 5,824,305, administration of isolated and purified stem bromelain protease is effective in the treatment of diseases and conditions including inflammation, atherosclerosis, cancer, and bacterial infections.

U.S. Pat. No. 8,409,840 to Müller discloses recombinant expressed cysteine proteases which are found in bromelain and uses thereof for treating cancer, atherosclerosis, bacterial infections, inflammations, thromboses and edema.

U.S. Pat. No. 8,754,045 to Livingston discloses a method of treating benign and malignant neoplasms which includes administering an effective amount of one or more debridement enzymes and a denaturant. Among the debridement enzymes, bromelain is listed.

U.S. Pat. No. 9,526,768 to Mai discloses compositions comprising proteolytic enzymes and fining agents and uses thereof for the treatment and/or prevention of cancer. According to U.S. Pat. No. 9,526,768, fining agents include carbon, gelatin, casein, albumen, chitosan, sodium alginate, alginic acid, and more.

European Pat. No. 1012304 discloses an isolated protein which has a molecular weight of about 27.45 kDa as determined by SDS-PAGE, an isoelectric point of 9.7 as determined by isoelectric focusing, wherein the isolated protein is a component of bromelain, for use in treating cancer.

European Pat. No. 2931296 discloses compositions containing one or more of the compounds contained in bromelain, or metabolites, salts, or solvates thereof, and at least one mucolytic agent, for treatment of diseases involving mucin, especially mucin secreting cancers, or diseases involving blood clots (thrombi). The mucolytic agents include N-acetyl cysteine, nacystelyn, mercapto-ethanolsulphonate, carbocysteine, dextran, heparin, and more.

US 2003/0026794 discloses methods for treating skin conditions by providing compositions containing at least one enzyme to selectively remove specific layers of skin. The conditions treatable by the methods include age-related conditions, infections, pigmentary disorders, follicular disorders, and hyperkeratotic disorders.

International Pub. No. WO 1996/000082 discloses use of bromelain in the treatment of diseases mediated by intracellular signal pathways which depend upon inositol phosphates, protein kinases and/or protein phosphatases, such as cancer and autoimmune diseases.

International Pub. No. WO 1998/038320 discloses a protein which is a component of bromelain, has a molecular weight of about 22.2 to 25.08 kDa as determined by SDS-PAGE, has an isoelectric point of about 3.8 to 4.79, and use thereof for treating cancer.

International Pub. No. WO 1998/038291 discloses a component of bromelain which contains proteins having molecular weights of about 15.07 kDa, 25.85 kDa and 27.45 kDa as determined by SDS-PAGE, has isoelectric points of 10.4 and 10.45, for use in treating cancer.

International Pub. No. WO 2000/014254 discloses a fraction of bromelain comprising proteins of molecular weights 23.9 kDa and 26.6 kDa when measured by SDS-PAGE or 23.459 kDa and 23.6576 kDa when measured by mass spectrometry; and use thereof for treating cancer.

International Pub. No. WO 2010/012438 discloses a heterologously expressed Bromelain inhibitor or Bromelain inhibitor precursor, wherein said Bromelain inhibitor or Bromelain inhibitor precursor is heterologously expressed in substantial amounts in soluble form, and use thereof for treating cancer.

International Pub. No. WO 2013/089803 discloses a method for treating and/or preventing various types of cancerous and neoplastic disorders in a mammal which comprises the administration thereto of an effective amount of Bromelain extract derived from Ananas Comosus. Administration was performed by intraperitoneal, oral or intravenous routes.

International Pub. No. WO 2019/161457 discloses a microsphere for delivery to a target area in a patient's body, the microsphere containing a mucin-affecting protease loaded therein, and adapted to elute the mucin-affecting protease in a sustained manner when exposed to physiological conditions. The mucin-affecting protease can be bromelain. Methods for treating a mucin-producing cancer comprising administering said microspheres are also disclosed.

International Patent Application Publication No. WO 2006/054309 to the applicant of the present invention discloses a debriding composition obtained from bromelain comprising most of the proteolytic enzymes present in bromelain and use thereof for debriding non-viable tissues.

International Patent Application Publication No. WO 2013/011514 to the applicant of the present invention discloses a proteolytic extract obtained from bromelain for the treatment of connective tissue diseases, in particular, Dupuytren's disease and Peyronie's disease.

International Patent Application Publication No. WO 2017/130204 to the applicant of the present invention discloses debriding compositions comprising a proteolytic enzyme mixture obtained from bromelain in the form of an aqueous gel useful for debridement and treatment of wounds.

International Patent Application Publication No. WO 2017/183018 to the applicant of the present invention discloses methods of debridement of chronic wounds comprising topically applying to a wound site a debriding formulation in the form of a hydrogel comprising a proteolytic enzyme mixture obtained from bromelain and a water-soluble gelling agent, the debriding formulation being applied to the wound site up to ten times over a period of up to four weeks, thereby achieving debridement of chronic wounds.

Rosenberg et al. describe off-label treatment experiment using Concentrate of Proteolytic Enzymes Enriched in Bromelain (CPEEB) for the destruction of six basal cell carcinoma lesions in three patients (The Open Dermatol. J. DOI: 10.2174/1874372202115010039, 2021, 15: 39-44)

There remains an unmet need for improved methods for treating skin lesions which are effective, rapid, and safe.

### SUMMARY OF THE INVENTION

The present invention provides methods and pharmaceutical formulations for treating skin lesions, the methods comprise topically applying to a skin lesion of a subject in need of such treatment a pharmaceutical formulation, in a form of a hydrogel, which comprises: (i) a proteolytic enzyme mixture obtained from bromelain comprising stem bromelain (EC 3.4.22.32); and (ii) a water-soluble gelling agent. The skin lesions to be treated are selected from the group consisting of non-melanoma skin cancers, pre-cancerous skin lesions, and benign skin lesions.

The present invention is based on the surprising findings that applying topically to basal cell carcinoma (BCC) lesions of human subjects a pharmaceutical formulation comprising: (i) a proteolytic enzyme mixture obtained from bromelain comprising stem bromelain (EC 3.4.22.32); (ii) a water-soluble gelling agent; and (iii) water, for up to ten applications, every other day, while maintaining the pharmaceutical formulation on the skin lesions for about 9-12 hours per application, resulted in tumor destruction within days, typically within less than two weeks, and in healing of the treated BCC lesions within about 4 weeks after cease of treatment with the pharmaceutical formulation. No hypertrophic scarring was observed.

The present invention discloses that the short-term topical treatment, e.g., five to six applications, with the pharmaceutical formulation for a duration of about two weeks, destroyed the common types of BCC lesions, e.g., superficial BCC lesions, infiltrative or invasive BCC lesions which include Morphea form BCC lesions, and nodular BCC lesions, and lead to healing of the BCC lesions so that essentially healthy skin appearance was achieved at the lesion site. It is now further disclosed that the methods of the present invention destroyed residual cancer cells of BCC as evaluated by Mohs micrographic surgery. The methods of the present invention caused merely local irritation and mild itching pain during the first two hours after the first applications, but these minor effects disappeared within two to four hours with no need of any medical treatment. Notably, the methods of the present invention are devoid of adverse effects common to radiotherapy or chemotherapy. Also, the methods of the present invention do not involve surgical or other physical destructive procedures. Therefore, the methods of the present invention are highly advantageous over known methods currently being used for destruction or removal of non-melanoma skin cancer lesions in human subjects.

It is now disclosed that under in vitro conditions the proteolytic enzyme mixture obtained from bromelain of the present invention was more cytotoxic to skin cancer cells than to normal or healthy epidermal cells, and that such cytotoxicity was higher than that exerted by purified stem bromelain. The formulations of the present invention therefore provide more selective means for treating non-melanoma skin cancer lesions than purified stem bromelain.

It is unexpectedly disclosed that the methods of the present invention prevented recurrence of non-melanoma skin cancer lesions, specifically BCC skin lesions, in human subjects, months after cease of treatment. The methods of the present invention therefore provide similar advantages as of surgical methods, i.e., delay or prevention of BCC recurrence, yet are devoid of complicated and expensive procedures, avoid damage and scarring of the healthy skin adjacent to the skin cancer lesions, and do not create cosmetic or esthetic defects; all of which are typical of surgical procedures.

Without being bound to any mechanism of action, the proteolytic enzyme mixture obtained from bromelain is shown herein to destroy the tumor mass, to eliminate residual cancer cells, and to promote recovery and restitution of the treated tissues ad integrum.

Thus, the methods of the present invention are highly efficient, rapid, safe, local, increase patient compliance, and cost effective.

According to a first aspect, the present invention provides a method of treating a skin lesion and delaying or preventing recurrence thereof comprising topically applying to a skin lesion of a subject in need of such treatment a therapeutically effective amount of a pharmaceutical formulation comprising:
(a) a composition in a dry or powdered form comprising:
   (i) a proteolytic enzyme mixture obtained from bromelain, also denoted throughout the specification active pharmaceutical ingredient (API), comprising stem bromelain (EC 3.4.22.32);
   (ii) a water-soluble gelling agent;
   (iii) optionally, an anti-aggregation agent;
   (iv) a pH adjusting agent; and
(b) water,
wherein, prior to use, the composition (a) is admixed with the water (b) to form the pharmaceutical formulation characterized by being a hydrogel having a pH ranging from about 6.0 to about 8.0,
wherein the proteolytic enzyme mixture obtained from bromelain is present in an amount ranging from about 0.5% (w/w) to about 10% (w/w) of the total weight of said pharmaceutical formulation, and
wherein the skin lesion is selected from the group consisting of non-melanoma skin cancers, pre-cancerous skin lesions, and benign skin lesions, thereby treating said skin lesion and delaying or preventing recurrence thereof.

According to some embodiments, the skin lesions are due to solar radiation.

According to additional embodiments, the non-melanoma skin cancer is selected from the group consisting of basal cell carcinoma (BCC), and squamous cell carcinoma (SCC).

According to further embodiments, the BCC is selected from the group consisting of superficial BCC, infiltrative BCC, nodular BCC, and combinations thereof.

According to yet further embodiments, the pre-cancerous skin lesion is selected from the group consisting of actinic keratoses, SCC in situ, a skin lesion due to Bowen's disease, actinic cheilitis, and moles.

According to still further embodiments, the benign skin lesion is seborrheic keratosis.

According to some embodiments, the recurrence of the skin lesion is delayed for at least 18 months. According to additional embodiments, when the skin lesion is BCC, the recurrence of said skin lesion is delayed for at least 24 months.

According to some embodiments, the pharmaceutical formulation is applied to the skin lesion for up to ten applications, or so long as said skin lesion is treated, and wherein said pharmaceutical formulation is maintained in contact with the skin lesion for at least about 4 hours up to 24 hours per application. According to further embodiments, the pharmaceutical formulation is applied to the skin lesion one to seven applications per week.

According to additional embodiments, the pharmaceutical formulation is applied to the skin lesion for up to seven applications, and wherein said pharmaceutical formulation is maintained in contact with said skin lesion for about 8 hours to about 12 hours per application. According to further embodiments, the pharmaceutical formulation is applied to the skin lesion one to six applications per week.

According to further embodiments, the pharmaceutical formulation is applied to the skin lesion for up to four consecutive weeks.

According to certain embodiments, the skin lesion is BCC and the pharmaceutical composition is applied to said skin lesion for five to seven, alternatively five or six, applications, every other day, for about 9-12 hours per application.

According to some embodiments, the method further comprises a step of washing and/or wiping the skin lesion after the at least about 4 to about 24 hours of contact of the pharmaceutical formulation with said skin lesion, prior to a subsequent application.

According to further embodiments, the method further comprises, following the up to ten topical applications of the pharmaceutical formulation, a step of topically applying to the treated skin lesion at least one healing composition.

According to yet further embodiments, the healing composition is selected from the group consisting of steroid ointments, fatty ointments, moisturizing creams, hydrophilic ointments, lotions, gels, sprays, aerosols, and combinations thereof.

According to some embodiments, the amount of the proteolytic enzyme mixture obtained from bromelain ranges from about 1% (w/w) to about 7% (w/w) of the total weight of the pharmaceutical formulation. According to a certain embodiment, the amount of the proteolytic enzyme mixture is of about 5% (w/w) of the total weight of the pharmaceutical formulation.

According to additional embodiments, the water-soluble gelling agent is selected from the group consisting of naturally occurring gelling agents, semi-synthetic gelling agents, and synthetic gelling agents. According to additional embodiments, the water-soluble naturally occurring gelling agent is a polysaccharide. According to further embodiments, the polysaccharide is a galactomannan, a glucomannan, or a combination thereof. According to still further embodiments, the galactomannan is guar gum present in an amount ranging from about 0.25% (w/w) to about 5% (w/w) of the total weight of the pharmaceutical formulation. According to additional embodiments, the water-soluble gelling agent is a polymer of acrylic acid, such as a carbomer. According to further embodiments, the carbomer is present in an amount ranging from about 0.5% (w/w) to about 2.5% (w/w), alternatively in an amount of about 1% (w/w) to about 1.5% (w/w).

According to further embodiments, the anti-aggregation agent is an oligosaccharide. According to further embodiments, the oligosaccharide is selected from the group consisting of lactose, sucrose, mannitol, glucose, and a combination thereof. According to still further embodiments, the oligosaccharide is lactose present in an amount ranging from about 10% (w/w) to about 25% (w/w) of the total weight of the pharmaceutical formulation.

According to yet further embodiments, the pH adjusting agent is potassium phosphate. According to further embodiments, the potassium phosphate is present in an amount ranging from about 2% (w/w) to about 10% (w/w) of the total weight of the pharmaceutical formulation. According to further embodiments, the potassium phosphate is a combination of potassium phosphate dibasic and potassium phosphate monobasic.

According to still further embodiments, the pH of the pharmaceutical formulation ranges from about 6.0 to about 7.0.

According to further embodiments, the water is present in an amount ranging from about 50% (w/w) to about 95% (w/w) of the total weight of the pharmaceutical formulation.

According to some embodiments, the hydrogel is a homogenous hydrogel. According to additional embodiments, the homogenous hydrogel has a viscosity in the range of about 2,000,000 centipoise (cP) to about 8,500,000 cP.

According to some embodiments, the composition is sterile. According to additional embodiments, the composition and water, each, are sterile. According to further embodiments, the pharmaceutical formulation is sterile.

According to additional embodiments, the pharmaceutical formulation further comprises an agent selected from the group consisting of anti-foaming agents, anti-oxidants, and preservatives.

According to further embodiments, the pharmaceutical formulation further comprises an active agent selected from the group consisting of anesthetic agents, anti-inflammatory agents, antibiotic agents, anti-fungal agents, analgesic agents, and healing promoting agents.

According to some embodiments, the pharmaceutical formulation useful for practicing the method of the present invention comprises:
(i) the proteolytic enzyme mixture obtained from bromelain in an amount ranging from about 0.5% (w/w) to about 10% (w/w), preferably from about 1% (w/w) to about 7% (w/w) of the total weight of the pharmaceutical formulation;
(ii) guar gum in an amount ranging from about 0.25% (w/w) to about 5% (w/w) of the total weight of the pharmaceutical formulation;
(iii) lactose in an amount ranging from about 10% (w/w) to about 25% (w/w) of the total weight of the pharmaceutical formulation;
(iv) potassium phosphate in an amount ranging from about 2% (w/w) to about 10% (w/w) of the total weight of the pharmaceutical formulation; and
(v) water in an amount to complete to 100% (w/w) of the total weight of the pharmaceutical formulation.

According to a certain embodiment, the pharmaceutical formulation useful for practicing the method of the present invention comprises:

| Ingredient | (%) w/w of formulation |
|---|---|
| Proteolytic enzyme | 5 |
| mixture obtained from | |
| bromelain (API) | |
| Guar gum | 3.5 |
| Lactose | 16.05 |
| Potassium phosphate | 2.5 |
| dibasic | |
| Potassium phosphate | 0.8 |
| monobasic | |
| PEG-3350 | 1 |
| Water for injection | 71.15 |

According to exemplary embodiments, the method of treating a skin lesion and delaying or preventing recurrence thereof comprising topically applying to a skin lesion of a subject in need of such treatment a therapeutically effective amount of a pharmaceutical formulation comprising:
(a) a composition in a dry or powdered form comprising:
   (i) a proteolytic enzyme mixture obtained from bromelain comprising stem bromelain (EC 3.4.22.32) present in an amount ranging from about 0.5% (w/w) to about 10% (w/w), preferably from about 1% (w/w) to about 7% (w/w) of the total weight of the pharmaceutical formulation;
   (ii) guar gum present in an amount ranging from about 0.25% (w/w) to about 5% (w/w) of the total weight of the pharmaceutical formulation;
   (iii) lactose in an amount ranging from about 10% (w/w) to about 25% (w/w) of the total weight of the pharmaceutical formulation;
   (iv) potassium phosphate in an amount ranging from about 2% (w/w) to about 10% (w/w) of the total weight of the pharmaceutical formulation; and
(b) water in an amount to complete to 100% (w/w) of the total weight of the pharmaceutical formulation,
wherein, prior to use, the composition (a) is admixed with the water (b) to form the pharmaceutical formulation characterized by being a hydrogel having a pH ranging from about 6.0 to about 8.0,
wherein the skin lesion is BCC, and
wherein the pharmaceutical formulation is applied to the BCC lesion up to ten times for up to 4 weeks, preferably for up to seven times for up to 2 weeks, thereby treating said BCC lesion and delaying or preventing recurrence thereof.

According to another aspect, the present invention provides a pharmaceutical formulation for use in treating a skin lesion, wherein the pharmaceutical formulation comprises:
(a) a composition in a dried or lyophilized form comprising:
   (i) a proteolytic enzyme mixture obtained from bromelain comprising stem bromelain (EC 3.4.22.32);
   (ii) a water-soluble gelling agent;
   (iii) optionally, an anti-aggregation agent;
   (iv) a pH adjusting agent; and
(b) water,
wherein, prior to use, the composition (a) is admixed with the water (b) to form said pharmaceutical formulation characterized by being a hydrogel having a pH ranging from about 6.0 to about 8.0,
wherein the amount of the proteolytic enzyme mixture obtained from bromelain ranges from about 0.5% (w/w) to about 10% (w/w) of the total weight of the pharmaceutical formulation, and
wherein the skin lesion is selected from the group consisting of non-melanoma skin cancers, pre-cancerous skin lesions, and benign skin lesions; according to the principles of the present invention.

These and other embodiments of the present invention will be better understood in relation to the figures, description, examples and claims that follow.

### BRIEF DESCRIPTION OF THE FIGURES

FIGS. 1A-F are photographs of an actinic Morphea type BCC lesion due to extensive sun exposure in a female subject, 35 years old. FIG. 1A shows the lesion before treatment and FIGS. 1B-1F show the lesion after application of the formulation of the present invention for 12 hours per each application, during the following treatment period: after the 1^{st} application (FIG. 1B); after the 2^{nd} application (FIG. 1C); after the 3^{rd} application (FIG. 1D); after the 4^{th} application (FIG. 1E); and after the 5^{th} application (FIG. 1F).

FIGS. 2A-F are photographs of the lesion of the subject of FIGS. 1A-F, during the recovery period: after 10 days (FIG. 2A); after 3 weeks (FIG. 2B); after 30 days/4 weeks (FIG. 2C); after 7 weeks (FIG. 2D); after 67 days/9 weeks (FIG. 2E); and after 11 weeks (FIG. 2F). It can be seen that healing and regeneration of the skin occurred at the 7^{th} week after cease of treatment with the formulation of the invention.

FIGS. 3A-F are photographs of an actinic invasive BCC lesion due to extensive sun exposure in a female subject, 35 years old. FIG. 3A shows the lesion before treatment and FIGS. 3B-3F show the lesion after application of the formulation of the present invention for 12 hours per each application, during the following treatment period: after the 1^{st} application (FIG. 3B); after the 2^{nd} application (FIG. 3C); after the 3^{rd} application (FIG. 3D); after the 4^{th} application (FIG. 3E); and after the 5^{th} application (FIG. 3F).

FIGS. 4A-F are photographs of the lesion of the subject of FIGS. 3A-F, during the recovery period: after 10 days (FIG. 4A); after 3 weeks (FIG. 4B); after 30 days/4 weeks (FIG. 4C); after 7 weeks (FIG. 4D); after 67 days/9 weeks (FIG. 4E); and after 11 weeks (FIG. 4F). It can be seen that a minimal scar was seen 4 weeks, and essentially no scar was left 9 weeks, after cease of treatment with the formulation of the invention.

FIGS. 5A-F are photographs of an actinic invasive BCC lesion due to extensive sun exposure in a female subject, 35 years old. FIG. 5A shows the lesion before treatment and FIGS. 5B-5F show the lesion after application of the formulation of the present invention for 12 hours per each application, during the following treatment period: after the 1^{st} application (FIG. 5B); after the 2^{nd} application (FIG. 5C); after the 3^{rd} application (FIG. 5D); after the 4^{th} application (FIG. 5E); and after the 5^{th} application (FIG. 5F).

FIGS. 6A-F are photographs of the lesion of the subject of FIGS. 5A-F, during the recovery period: after 10 days (FIG. 6A); after 3 weeks (FIG. 6B); after 30 days/4 weeks (FIG. 6C); after 7 weeks (FIG. 6D); after 67 days/9 weeks (FIG. 6E); and after 11 weeks (FIG. 6F). It can be seen that a minimal scar was seen 4 weeks, and no scar was left 9 weeks, after cease of treatment with the formulation of the invention.

FIGS. 7A-G are photographs of an actinic nodular BCC lesion due to extensive sun exposure in a male subject, 72 years old. The lesion had a diameter of 6 mm and appeared on the subject's left arm 3 years prior to treatment. FIG. 7A shows the lesion before treatment and FIGS. 7B-7G show the lesion after application of the formulation of the present invention for 12 hours per each application, during the following treatment period: after the 1^{st} application (FIG. 7B); after the 2^{nd} application (FIG. 7C); after the 3^{rd} application (FIG. 7D); after the 4^{th} application (FIG. 7E); after the 5^{th} application (FIG. 7F); and after the 6^{th} application (FIG. 7G). It can be seen that a significant effect on the lesion took place after 3-4 applications.

FIGS. 8A-H are photographs of the lesion of the subject of FIGS. 7A-G, during the recovery period: after 1 day (FIG. 8A); after 2 days (FIG. 8B); after 4 days (FIG. 8C); after 8 days (FIG. 8D); after 32 days (FIG. 8E); after 41 days (FIG. 8F); after 50 days (FIG. 8G); and after 60 days (FIG. 811). It can be seen that no scar was left 6 weeks after cease of treatment with the formulation of the invention.

FIGS. 9A-F are photographs of an actinic nodular BCC lesion due to extensive sun exposure in a male subject, 72 years old. The lesion had a diameter of 6 mm and appeared on the subject's left leg 3 years prior to treatment. FIGS. 9A-9F show the lesion after application of the formulation of the present invention for 12 hours per each application, during the following treatment period: after the 1^{st} application (FIG. 9A); after the 2^{nd} application (FIG. 9B); after the 3^{rd} application, with ZnO₂ to protect the surrounding skin (FIG. 9C); after the 3^{rd} application, without ZnO₂ (FIG. 9D); after the 4^{th} application (FIG. 9E); and after the 5^{th} application (FIG. 9F). It can be seen that a significant effect on the lesion took place after 3-4 applications.

FIGS. 10A-H are photographs of the lesion of the subject of FIGS. 9A-F, during the recovery period: post washing (FIG. 10A); after 1 day (FIG. 10B); after 2 days (FIG. 10C); after 9 days (FIG. 10D); after 11 days (FIG. 10E); after 41 days/5 weeks (FIG. 10F); after 6 weeks (FIG. 10G); and after 8 weeks (FIG. 10H). It can be seen that a minimal pigmentation/scarring was seen 11 days, and no scar was left 5 weeks after cease of treatment with the formulation of the invention.

FIGS. 11A-C show the effect of the proteolytic enzyme mixture obtained from bromelain (API) and of pure stem bromelain on the viability of normal human epidermal keratinocytes (HEK) and on squamous cell carcinoma (SCC) and basal cell carcinoma (BCC) cell lines under in vitro conditions. The cells were incubated in the absence or presence of increasing concentrations of the API or pure stem bromelain for 24 hours. Viability was determined by MTT; The symbol (*) defines p<0.05 for significant reduction as compared to cells treated with vehicle only. The symbol (#) defines p<0.05 for higher or lower viability as compared to control HEK cells.

FIGS. 12A-C show the effect of the API and of pure stem bromelain on the viability of normal HEK cells and on SCC and BCC cell lines under in vitro conditions. The cells were incubated in the absence or presence of increasing concentrations of the API or pure stem bromelain for 30 hours. Viability was determined by MTT; The symbol (*) defines p<0.05 for significant reduction as compared to cells treated with vehicle only. The symbol (#) defines p<0.05 for higher or lower viability as compared to control HEK cells.

FIG. 13A-F show the induction of apoptosis by the API or pure stem bromelain in normal BEK cells and in SCC and BCC cell lines under in vitro conditions. The cells were incubated in the absence or presence of increasing concentrations of the API or pure stem bromelain for 30 hours. FIGS. 13A-C show cell apoptosis as determined by caspase-3 activity assay. FIGS. 13D-F show cell apoptosis caused by 50 μg/ml of the API or pure stem bromelain which was normalized to viability. C—non-treated cells; V—Vehicle treated cells; B— bromelain; CPEEB—Concentrate of Proteolytic Enzymes Enriched in Bromelain, designated herein as the API. The symbol (*) defines p<0.05 for significant reduction as compared to control non-treated cells; The symbol (#) defines p<0.05 for lower apoptosis in comparison to the respective control HEK cells.

FIGS. 14A-C show the cell proliferation rate in normal HEK cells and in SCC and BCC cell lines under in vitro conditions. The cells were incubated in the absence or presence of increasing concentrations of CPEEB, designated herein as the API, or pure stem bromelain for 30 hours. Proliferation was determined by BrdU incorporation; The symbol (*) defines p<0.05 for significant reduction from control non-treated cells.

### DETAILED DESCRIPTION OF THE INVENTION

The present invention provides methods and formulations for use in treating a skin lesion due to non-melanoma skin cancers and/or pre-cancerous skin lesions and/or benign skin lesions, the methods comprise topically applying to a skin lesion site of a subject in need of such treatment a therapeutically effective amount of a pharmaceutical formulation in the form of a hydrogel, wherein the pharmaceutical formulation comprises: (i) a proteolytic enzyme mixture obtained from bromelain comprising stem bromelain (EC 3.4.22.32); and (ii) a water-soluble gelling agent.

### Pharmaceutical Formulations

The present invention provides a pharmaceutical formulation comprising a proteolytic enzyme mixture obtained from bromelain as an active ingredient and various excipients.

The term “proteolytic enzyme mixture obtained from bromelain” as used throughout the specification and claims refers to an enzymatic preparation partially purified from bromelain.

The term “bromelain” refers to an extract, predominantly proteinaceous, derived from the stems of pineapple plants which can be purchased commercially.

The proteolytic enzyme mixture obtained from bromelain (previously termed Debrase® or NexoBrid®) and the preparation thereof are disclosed in WO 2006/054309, WO 2013/011514 and WO 2017/183018, the content of which is incorporated by reference as if fully set forth herein. The proteolytic enzyme mixture obtained from bromelain comprises stem bromelain (EC 3.4.22.32). The proteolytic enzyme mixture can further comprise ananain (EC 3.4.22.31). The proteolytic enzyme mixture can further comprise one or more of the cysteine protease precursors of bromelain such as, for example, stem bromelain (EC 3.4.22.31) precursor, fruit bromelain (EC 3.4.22.33) precursor, and ananain (EC 3.4.22.31) precursor. The proteolytic enzyme mixture can further comprise cysteine protease fragments (see, for example, WO 2006/054309), a jacalin-like lectin, and/or bromelain inhibitors. According to a certain embodiment, the proteolytic enzyme mixture obtained from bromelain comprises stem bromelain (EC 3.4.22.32), ananain (EC 3.4.22.31), a cysteine protease precursor of bromelain, and a jacalin-like lectin.

The proteolytic enzyme mixture can be obtained by the procedure disclosed in WO 2013/011514. As the last step of the preparation, the proteolytic mixture is lyophilized and stored as a lyophilized powder until use.

The proteolytic enzyme mixture is highly stable and can be stored at 2-8° C. for long periods of time, e.g., up to three years. After this period of time, the proteolytic enzyme mixture maintains at least 90% of the original debriding activity determined immediately after the preparation process.

The proteolytic enzyme mixture obtained from bromelain is denoted throughout the specification and claims as the active pharmaceutical ingredient (API). According to the invention, the amount of API in the pharmaceutical formulation ranges from about 0.5% (w/w) to about 10% (w/w) of the total weight of the pharmaceutical formulation. According to additional embodiments, the amount of API ranges from about 1% (w/w) to about 7% (w/w), such as of about 1% (w/w), 2%, 3%, 4%, 5%, 6%, 7%, or any amount in between, of the total weight of the pharmaceutical formulation. According to a certain embodiment, the amount of API is of about 5% (w/w) of the total weight of the pharmaceutical formulation.

The terms “dry”, “dried”, “lyophilized” or “powdered” composition as used interchangeably throughout the specification and claims refer to the composition which contains water in an amount of no more than about 5% (w/w) of the total weight of the composition, alternatively water is present in an amount of no more than about 3%, 2%, 1%, 0.5%, or further alternatively no more than about 0.1% (w/w) of the total weight of the composition. According to a certain embodiment, the composition is devoid of water.

The term “hydrogel” as used herein refers to an aqueous composition capable of maintaining a gel-like form.

The term “homogenous” hydrogel as used herein means a hydrogel having uniform viscosity (e.g., well mixed throughout).

The excipients of the pharmaceutical formulation are all pharmaceutically acceptable. The term “pharmaceutically acceptable” means approved by a regulatory agency of the Federal or a state government or listed in the U. S. Pharmacopeia or other generally recognized pharmacopeia for use in humans.

The term “about” refers to a value which is 10% above or below the indicated value.

According to some embodiments, the excipients of the pharmaceutical formulation are water-soluble. The term “water soluble” refers to an agent which typically has solubility in water in the range of 1 gr/ml to 1 gr/30 ml at room temperature, i.e., at about 22° C. to about 25° C.

The water-soluble gelling agent can be a naturally occurring gelling agent, a semi-synthetic gelling agent, and a synthetic gelling agent.

The water-soluble naturally occurring gelling agent include, but are not limited to, water-soluble naturally occurring polysaccharides such as, for example, galactomannans, glucomannans, starches, agar, pectins, carrageenans, or a combination thereof. Each possibility represents a separate embodiment. Non-limiting examples of galactomannans and glucomannans are guar gum, locust bean gum, xanthan gum, gum acacia, gum tragacanth, gellan gums, and mixtures thereof. Each possibility represents a separate embodiment. According to a certain embodiment, the water-soluble naturally occurring gelling agent is a galactomannan, such as guar gum.

Other water-soluble naturally occurring gelling agents include, for example collagens, glycosaminoglycans such as, for example, chondroitin sulfate, dermatan sulfate, and heparan sulfate, proteoglycans, fibronectins, and laminins.

Semi-synthetic gelling agents include, but are not limited to, cellulose ethers (e.g. hydroxyethyl cellulose, methyl cellulose, carboxymethyl cellulose, hydroxy propylmethyl cellulose), polyvinylpyrrolidone, polyvinylalcohol, hydroxypropyl guar gum, and the like.

The synthetic gelling agents include, but are not limited to, polymers of acrylic acid, e.g., carbomer polymers, carboxyvinyl polymers, polyvinylpyrrolidone, polyvinyl acetate polymers, polyvinyl chloride polymers, polyvinylidene chloride polymers, and the like. According to some embodiments, the synthetic gelling agent is a carbomer, such as Carbopol® 980 NF.

The pharmaceutical formulation can further comprise at least one excipient selected from the group consisting of anti-aggregation agents and pH adjusting agents.

The anti-aggregation or anti-agglomeration agent suitable for practicing the present invention is any known anti-aggregation agent capable of reducing or preventing the aggregation of the proteolytic enzymes present in the proteolytic enzyme mixture obtained from bromelain. Examples of anti-aggregation agents include water-soluble oligosaccharides such as, for example, lactose, sucrose, mannitol, sorbitol, and glucose, and amino acids such as arginine, histidine, and the like. Each possibility represents a separate embodiment. According to a certain embodiment, the anti-aggregation agent is lactose.

The pH adjusting agent preferably has a pKa of above 6.0. In some embodiments, the pH adjusting agent can be any known pH adjusting agent such as, for example, potassium phosphate, potassium carbonate, sodium carbonate, and sodium phosphate. According to some embodiments, the pH adjusting agent is a combination of potassium phosphate monobasic and potassium phosphate dibasic present in an amount ranging from about 2% (w/w) to about 10% (w/w) of the total weight of the pharmaceutical formulation.

The pharmaceutical formulation can further comprise an anti-foaming agent. Anti-foaming agents are known in the art and include, but are not limited to, polyethylene glycols, e.g., PEG-1450, PEG-3350, and the like. The pharmaceutical formulation can further comprise a preservative such as, for example, benzyl alcohol, parabens, methyl- or propylhydroxybenzoates; and/or an anti-oxidant such as, for example, ascorbic acid, dihydroquinone, butylated hydroxytoluene and dithiothreitol.

The pharmaceutical formulation can further comprise one or more anesthetic agents, antibacterial agents, antifungal agents, anti-inflammatory agents, analgesic agents, and/or promoting healing agents.

The anesthetic agents include, but are not limited to, amethocaine (tetracaine), lignocaine (lidocaine), xylocaine, bupivacaine, prilocaine, ropivacaine, benzocaine, mepivocaine, cocaine. Each possibility represents a separate embodiment.

The antibacterial agents include, but are not limited to, amanfadine hydrochloride, amanfadine sulfate, amikacin, amikacin sulfate, amoglycosides, amoxicillin, ampicillin, amsamycins, bacitracin, beta-lactams, candicidin, capreomycin, carbenicillin, cephalexin, cephaloridine, cephalothin, cefazolin, cephapirin, cephradine, cephaloglycin, chilomphenicols, chlorhexidine, chloshexidine gluconate, chlorhexidine hydrochloride, chloroxine, chlorquiraldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erhmycin stearate, farnesol, floxacillin, gentamicin, gentamicin sulfate, gramicidin, giseofulvin, haloprogin, haloquinol, hexachlorophene, iminocylcline, iodochlorhydroxyquin, kanamycin, kanamycin sulfate, lincomycin, lineomycin, lineomycin hydrochloride, macrolides, meclocycline, methacycline, methacycline hydrochloride, methenine, methenamine hippurate, methenamine mandelate, methicillin, metonidazole, miconazole, miconazole hydrochloride, minocycline, minocycline hydrochloride, mupirocin, nafcillin, neomycin, neomycin sulfate, netimicin, netilmicin sulfate, nitrofurazone, norfloxacin, nystatin, octopirox, oleandomycin, orcephalosporins, oxacillin, oxyteacline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, silver salts, spectinomycin, spiramycin, struptomycin, sulfonamide, tetracyclines, tetracycline, tobramycin, tobramycin sulfate, triclocarbon, triclosan, trimethoprim-sulfamethoxazole, tylosin, vancomycin, and yrothricin. Each possibility represents a separate embodiment.

The antifungal agents include, but are not limited to, nystatin, clotrimazole, miconazole, ketoconazole, fluconazole, thiabendazole, econazole, clomidazole, isoconazole, tiabendazole, tioconazole, sulconazole, bifonazole, oxiconazole, fenticonazole, omoconazole, sertaconazole, and flutrimazole. Each possibility represents a separate embodiment.

The anti-inflammatory agents can be non-steroidal anti-inflammatory agents, steroidal anti-inflammatory agents, or a combination thereof. Non limiting examples of non-steroidal anti-inflammatory agents include oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Extracts of these non-steroidal anti-inflammatory agents may also be employed. Each possibility represents a separate embodiment.

Non-limiting examples of steroidal anti-inflammatory agents include corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocorisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, and triamcinolone. Each possibility represents a separate embodiment.

Analgesic agents include, but are not limited to, codeine, hydrocodone, oxycodone, fentanyl, and propoxyphene. Each possibility represents a separate embodiment.

Agents which promote healing include, but are not limited to, hyaluronic acid, zinc oxide, and any healing agent known in the art.

The viscosity of the gel formulations of the present invention can be measured by any known means. According to some embodiments, an absolute viscometer with plate plate geometry can be used to determine the viscosity of the gel formulations described herein.

Alternatively, the viscosity can be determined by absolute viscosity measurements using cone plate geometry or by a Brookfield (spindle and cup) viscometer. The viscosity values referred to herein are all measured at 22° C.-25° C.

According to the principles of the present invention, the pharmaceutical formulation (a) which is present in a dry or powdered form and the water (b) can be placed in a first compartment and a second compartment, respectively, of a single container or can be placed in two separate containers. Before use, the composition (a) and the water (b) are admixed to form the pharmaceutical formulation.

The pharmaceutical formulations of the present invention are of low bacterial bioburden, and therefore the formulations of the present invention reduce the risk of further contaminating the lesion site. According to some embodiments, the pharmaceutical formulations are sterile.

According to some embodiments, the pharmaceutical formulation comprises:
(a) a composition in a dried or powdered form comprising:
   (i) a proteolytic enzyme mixture obtained from bromelain, denoted herein API, comprising stem bromelain (EC 3.4.22.32);
   (ii) a water-soluble gelling agent;
   (iii) optionally, an anti-aggregation agent;
   (iv) a pH adjusting agent; and
(b) water,
wherein the composition (a) being admixed with the water (b) to form a pharmaceutical formulation characterized by being a hydrogel, preferably a homogenous hydrogel, having a pH ranging from about 6.0 to about 8.0, and wherein the amount of API in the pharmaceutical formulation ranges from about 0.5% (w/w) to about 10% (w/w) of the total weight of the pharmaceutical formulation.

According to some embodiments, the pharmaceutical formulation comprises guar gum, said pharmaceutical formulation has a viscosity in the range of about 2,000,000 cP to about 8,500,000 cP at 22-25° C. According to further embodiments, the pharmaceutical formulation comprising guar gum has a viscosity in the range of about 2,400,000 cP to about 6,200,000 cP at 22-25° C.

According to some embodiments, the pharmaceutical formulation comprises:
(a) a composition in a dried or powdered form, present in a first compartment of a container or in a first container, the composition comprising:
   (i) a proteolytic enzyme mixture obtained from bromelain comprising stem bromelain (EC 3.4.22.32) in an amount of about 1% (w/w) to about 7% (w/w), preferably in an amount of about 5% (w/w), of the total weight of the pharmaceutical formulation;
   (ii) guar gum in an amount ranging from about 0.25% (w/w) to about 5% (w/w) of the total weight of the pharmaceutical formulation;
   (iii) lactose in an amount ranging from about 10% (w/w) to about 25% (w/w) of the total weight of the pharmaceutical formulation;
   (iv) a pH adjusting agent; and
(b) water in an amount ranging from about 55% (w/w) to about 90% (w/w), present in a second compartment of the container or in a second container.

According to some embodiments, the pharmaceutical formulation of the present invention is prepared by the following steps:
(a) obtaining a composition in a dry or powdered form which comprises:
   (i) the proteolytic enzyme mixture obtained from bromelain;
   (ii) the water-soluble gelling agent;
   (iii) optionally, an anti-aggregation agent;
   (iv) a pH adjusting agent; and
(b) admixing, prior to use, the composition (a) with water to form the pharmaceutical formulation characterized by being a hydrogel, preferably a homogenous hydrogel.

According to some embodiments, the hydrogel has a viscosity in the range of about 2,000,000 centipoise (cP) to about 8,500,000 cP, and a pH ranging from about 6.0 to about 8.0.

It is to be understood that the pharmaceutical formulations of the present invention are formulated as gels, i.e., hydrogels, and as such are applied onto the skin lesion site. According to some embodiments, the formulations are devoid of adhesive agents, and thus the formulations are non-adhesive. According to additional embodiments, the formulations are devoid of gelatin, casein, albumin, chitosan, sodium alginate, alginic acid, heparin, N-acetyl cysteine, nacystelyn, mercapto-ethanolsulphonate, carbocysteine, and dextran. According to further embodiments, the pharmaceutical formulations of the present invention are devoid of denaturants, thus avoiding the denaturation effect of denaturants which can harm the activity of the proteolytic enzymes of the pharmaceutical formulation of the present invention.

### Uses of the Pharmaceutical Formulation

The present invention provides methods for treating non-melanoma skin cancers and/or pre-cancerous skin lesions and/or benign skin lesions, the methods comprise a treatment step of topically applying to a lesion site of a subject in need of such treatment a therapeutically effective amount of a pharmaceutical formulation which comprises: (i) a proteolytic enzyme mixture obtained from bromelain comprising stem bromelain (EC 3.4.22.32); (ii) a water-soluble gelling agent; and (iii) water, according to the principles the present invention. The methods of the present invention can further delay or prevent skin lesion recurrence.

The terms “treating”, “treat”, “treatment” and the like, are meant to include reducing the size of a skin lesion, the color of a skin lesion, or any other visual characteristic of a skin lesion, or destroying or eliminating a skin lesion. These terms also include slowing or arresting the growth of a skin lesion. The methods of the present invention reduce the size, color, or any other visual characteristic of a skin lesion by at least about 10%, 20%, 30%, 40%, 50%, 60%, or by at least 70% as compared to the size and/or color and/or any other visual characteristic of the skin lesion prior to treatment with the formulation of the present invention. According to certain embodiments, the methods of the present invention reduce the size, color, or any other visual characteristic of a skin lesion by at least about 80% or 90%, and preferably by 100% as compared to the size and/or color and/or any other visual characteristic of the skin lesion prior to treatment with the formulation of the present invention. The appearance of a skin lesion may be evaluated visually, such as, but not limited to, by the subject and/or a physician.

The term a “skin lesion” refers to an area of variation in skin color and/or texture. According to some embodiments, a skin lesion to be treated includes a border of some mm, such as 1-10 mm, of apparently healthy tissue surrounding the skin lesion. According to some embodiments, the skin lesion appears in a form of an elevated skin area, lump, blister, multicolor skin area, scale, crust, wart-like, and a combination thereof. Each possibility represents a separate embodiment. The clinical appearance of a skin lesion is diverse and includes, but not limited to, nodular, cystic, ulcerated (‘rodent ulcer’), superficial, morphoeic (sclerosing), keratotic, and pigmented. Ulceration is especially common in large tumors, in long-standing or aggressive skin lesions.

The term “therapeutically effective amount” is that amount of the proteolytic enzyme mixture obtained from bromelain which is sufficient to provide a beneficial effect to the subject to which the composition is applied. Such a beneficial effect includes, for example, reducing the size of a skin lesion, destroying or eliminating the skin lesion, arresting skin lesion growth, reducing the rate of skin lesion growth, delaying or preventing skin lesion recurrence, and/or preventing metastasis.

The term “recurrence” of a skin lesion refers to the re-emergence, re-appearance, re-growth of a skin lesion of the same type in the same location, following a period during which the original skin lesion has been disappeared or during which the growth of the original skin lesion has been arrested or inhibited.

The term “following cease of treatment” as used herein means after treatment with the pharmaceutical formulation of the present invention is stopped.

The methods of the present invention are particularly useful for treating a skin lesion selected from the group consisting of non-melanoma cutaneous (skin) cancers, pre-cancerous skin lesions, and benign skin lesions, typically due to solar exposure and/or indoor tanning. Each possibility represents a separate embodiment of the invention.

The non-melanoma skin cancer to be treated by the methods of the present invention is selected from the group consisting of basal cell carcinoma (BCC), squamous cell carcinoma (SCC), Merkel cell carcinoma, and atypical fibroxanthoma. Each possibility represents a separate embodiment. According to certain embodiments, the non-melanoma skin cancer to be treated by the methods of the present invention is BCC or SCC.

BCC can appear as a painless raised area of skin, which may be shiny with small blood vessels running over it. It may also present as a raised area with ulceration. Basal-cell carcinoma grows slowly and can damage or erode the tissue around it, but it is unlikely to spread to distant areas or result in death.

Basal-cell carcinoma can broadly be divided into three groups, based on the growth patterns:
(1) Superficial basal-cell carcinoma, formerly referred to in-situ basal-cell carcinoma, is characterized by a superficial proliferation of neoplastic basal-cells.
(2) Infiltrative basal-cell carcinoma, which also encompasses morphea form and micronodular basal-cell carcinoma, typically penetrates into deeper layers of the skin.
(3) Nodular (also termed large nodular) basal-cell carcinoma includes most of the remaining categories of basal cell carcinoma including, but not limited to, Rodent ulcer (also known as “Jacob's ulcer”), polypoid basal cell carcinoma, and pore-like basal cell carcinoma.

It is to be understood that a BCC skin lesion can exhibit features of two BCC groups. The present invention therefore encompasses all BCC skin lesions known in the art.

SCC of the skin, also known as cutaneous squamous-cell carcinoma (cSCC), is less common than BCC but it is more aggressive than BCC. SCC is more likely to spread to distant areas in the body. SCC can appear in every part of the body, and can also develop on lips, vulva and penis; often it originates from burn scars or skin ulcers, and appears as a superficial lesion that easily bleeds. In some cases, an ulceration develops, with thick crater-like borders; in other cases, the lesion is covered by a horny layer. One form of SCC is the keratoacanthorma, in which a bulge or thick mass, often ulcerated, is formed in the parts of the body exposed to the sun.

The terms a “pre-cancerous” or “pre-malignant” skin lesion is defined as a skin lesion comprising abnormal cells that could turn into cancerous or malignant cells, but which, by themselves, are not invasive but may evolve into BCC or SCC, inflicting pain, itch, and even bleeding. The pre-cancerous cells are therefore associated with an increased risk of developing into cancer.

Pre-cancerous skin lesions include, but are not limited to, actinic keratoses, SCC in situ, a skin lesion due to Bowen's disease, actinic cheilitis, moles, and combinations thereof. Each possibility represents a separate embodiment. Also included as a pre-cancerous skin lesion “erythroplasia of Queyrat”, a superficial form of in-situ SCC of male genitals.

Actinic keratoses (also known as solar keratoses) is a pre-cancerous skin condition characterized by having thick, scaly, and/or crusty skin lesions, where epidermal keratinocytes are induced to grow by extensive ultraviolet (UV) light exposure. If left untreated, the actinic keratoses can turn into SCC.

SCC in situ and Bowen's disease are often used interchangeably and refer to a pre-cancerous forn of cSCC, i.e., it is predominantly non-invasive and it has only a 10% chance of developing into SCC. The main sign is a red, scaly patch on the skin. It affects the squamous cells which constitute the outermost layer of skin. While SCC in situ refers to a single skin lesion, Bowen's disease refers to multiple skin lesions of SCC in situ.

Actinic Cheilitis (Farmer's Lip) is a pre-cancerous lip condition induced by extensive UV light exposure of the lip's epithelium which is a form of actinic keratoses occurring on the lips. If left untreated, the actinic cheilitis can turn into SCC.

The term a “benign” skin lesion refers to a skin lesion that is not cancerous, it does not invade nearby tissues, and it does not spread to other pails of the body.

According to some embodiments, the pharmaceutical formulation of the present invention can be useful in treating benign skin lesions including, but not limited to, seborrheic keratoses.

The therapeutically effective amount of the proteolytic enzyme mixture obtained from bromelain for treating a skin lesion will vary according to factors known in the art including, but not limited to, the size, location, and histological type of the skin lesion being treated; the intended dosing regimen; and the clinical state of the subject to be treated. Accordingly, those of ordinary skill in the art can readily determine the appropriate amount with due consideration of such factors.

According to some embodiments, the method of treatment of the present invention comprises topically applying the pharmaceutical formulation onto a skin lesion site for up to 10 applications, wherein each application is of at least 4 hours up to about 24 hours. According to some embodiments, the treatment of a skin lesion lasts for up to four weeks.

According to some embodiments, the pharmaceutical formulation is topically applied to a skin lesion of a subject in need of such treatment for 7-10 applications. According to additional embodiments, the pharmaceutical formulation is topically applied to a skin lesion of a subject in need of such treatment for 2 to 9 or 2 to 8 applications. According to further embodiments, the pharmaceutical formulation is topically applied for 2 to 7 applications. According to yet still further embodiments, the pharmaceutical formulation is topically applied for 5 to 7 applications, alternatively for 5 or 6 applications.

According to some embodiments, the pharmaceutical formulation is maintained in contact with the lesion site for at least about 4 hours to about 24 hours per application. According to further embodiments, the pharmaceutical formulation is maintained in contact with the lesion site for at least 6, 8, 10, 12, 14, 16, 18, 20, 22, up to about 24 hours per application. Each possibility is a separate embodiment. According to still further embodiments, the pharmaceutical formulation is maintained in contact with the lesion site for about 8 to 12 hours per application. According to some embodiments, the pharmaceutical formulation is maintained in contact with the lesion site for about 9 to 12 hours per application.

According to some embodiments, the pharmaceutical formulation can be applied to a lesion site for up to 10 applications or as many applications as required so long as the lesion site is treated or disappears, wherein the pharmaceutical formulation is maintained in contact with the lesion site for at least 4 hours up to about 24 hours per application. Thus, the pharmaceutical formulation can be applied daily for up to 10 consecutive days so as to be maintained in contact with the lesion site for about 8 hours to about 12 hours per application or can be applied 1, 2, 3, 4, 5, 6, 7, 8, 9, or 10 applications for about 8 hours to about 12 hours per application with a halt of application in between of one day or more as required. Alternately, the pharmaceutical formulation can be applied 1 to 6 times per week, alternatively 1 to 3 times per week, for up to 4 consecutive weeks, so as to be maintained in contact with the lesion site for about 9 hours up to about 12 hours per application.

After the contact of the pharmaceutical formulation with the lesion site for the indicated application time, such as for at least 4 hours treatment, preferably for 12 hours treatment, or for any application time as disclosed herein above, the lesion site can be washed and wiped. Thus, the methods of the present invention can further comprise a step of washing the lesion site after said contact, prior to a subsequent application of the pharmaceutical formulation. If a halt of application is performed, the lesion site can be covered with a recovery or healing composition, preferably a fatty ointment, e.g., petrolatum-based ointment, or any moisturizing cream and/or ointment.

According to some embodiments, the methods of the present invention can further comprise a step of covering the pharmaceutical formulation with an occlusive layer or dressing to contain, maintain or hold the pharmaceutical formulation at the lesion site.

According to some embodiments, the method of the present invention can further comprise a step of protecting the surrounding skin at the lesion edges during treatment. The step of protecting the surrounding skin comprises applying to said surrounding skin an adhesive ointment, paste, cream, or the like, which serves as an adhesive barrier to contain the pharmaceutical formulation of the invention and to adhere to an occlusive dressing.

According to some embodiments, after the 1 to 10 applications of the pharmaceutical formulation, denoted herein the “treatment period”, the method of treating a skin lesion of the present invention can further comprise topically applying to the treated skin lesion site at least one healing composition, also denoted throughout the specification and claims a “recovery composition”, thereby promoting healing of said skin lesion.

The term “healing of a skin lesion” refers to a process of regeneration of the epidermal cell layers left after the skin lesion disappears, thus promoting restoration of the treated skin lesion site, thereby preventing the formation of hypertrophic scarring. Preferably, the site of the skin lesion which undergoes healing acquires skin appearance essentially similar, preferably identical, to the appearance of the healthy skin adjacent to the skin lesion.

According to some embodiments, the topical application of at least one healing or recovery composition following cease of treatment can be performed daily or several times per week, for a duration of at least one week, at least two weeks, at least three weeks, or for about four weeks, or so long as the skin lesion disappears or the site of the skin lesion heals; this period is denoted, interchangeably, throughout the specification and claims the “healing period” or the “recovery period”.

According to some embodiments, the at least one healing or recovery composition can be applied once a day or several times per day for a duration of up to two months. According to some embodiments, the at least one healing or recovery composition can be applied once a day for a duration of about one month. According to some embodiments, the healing or recovery period lasts about 4 weeks. According to further embodiments, the healing or recovery period lasts up to 6 weeks. According to yet further embodiments, the healing or recovery period lasts up to 2, alternatively up to 3 months.

The healing or recovery period can include topical application of one or more healing or recovery compositions to the skin lesion site.

The healing or recovery compositions can be selected from the group consisting of steroid ointments, fatty ointments, moisturizing creams, hydrophilic ointments, lotions, gels, sprays, aerosols, and combinations thereof. Each possibility represents a separate embodiment. According to some embodiments, the steroid ointment comprises hydrocortisone, betamethasone, prednisolone, or derivatives or combinations thereof. Each possibility represents a separate embodiment. According to some embodiments, the fatty ointment is selected from the group consisting of petrolatum-based ointments, mineral oil-based ointments, lanolin alcohol-based ointments, glycerin based semi-solid, and combinations thereof. Each possibility represents a separate embodiment. According to additional embodiments, the healing or recovery composition can comprise zinc oxide, hyaluronic acid, or any agent known to promote healing of a wound.

According to some embodiments, the methods of the present invention achieve complete healing of a skin lesion. The term “complete healing of a skin lesion” refers to disappearance of the skin lesion and restoration of a healthy skin appearance. Thus, the methods of the present invention can achieve complete healing of a skin lesion about 1 month, about 2 months, alternatively about 3 months after cease of treatment with the pharmaceutical formulation of the present invention.

The methods of the present invention delay or prevent recurrence of a skin lesion. The methods of the present invention decrease the rate of recurrence of a skin lesion such that the time of recurrence increases by at least 1.25-fold, 1.5, 1.75, 2, 2.5, 5, or by at least 10-fold as compared to the time of recurrence when the skin lesion is left untreated. According to some embodiments, the pharmaceutical formulation is administered for a desired duration of time. Then, treatment with the pharmaceutical formulation is stopped and the skin lesion is monitored. As contemplated herein, the pharmaceutical formulation of the present invention is capable of preventing or delaying skin lesion recurrence following cease of treatment with the pharmaceutical formulation described herein for a period of at least 12 months, alternatively for a period of at least 18, or for at least 24 months. According to a certain embodiment, the method of the present invention delays recurrence of a skin lesion such as BCC for at least 24 months.

According to some embodiments, there is provided a pharmaceutical formulation for use in the treatment of non-melanoma skin cancers and/or pre-cancerous skin lesions and/or benign skin lesion, wherein the pharmaceutical formulation is present in the form of a hydrogel comprising: (i) a proteolytic enzyme mixture obtained from bromelain comprising stem bromelain (EC 3.4.22.32); (ii) a water-soluble gelling agent; and (iii) water, according to the principles of the present invention.

The present invention encompasses combination therapy wherein the methods of the present invention can be performed prior to and/or after known lesion removal methods, such as, surgery (excision), excision followed by histology of the excised tissue (excisional biopsy and MOHS micrographic surgery), ablation by heat (Electrodessication and curettage, cautery, lasers), cryo-therapy (cryosurgery, freezing), photodynamic therapy (PDT), mechanical (scraping), chemical mummification and chemotherapy (5FU cream), Aldera (imiquimod) and Picato (ingenol mebutate). Thus, according to some embodiments, the methods of the present invention can be performed prior to a surgical lesion removal. Alternatively, the methods of the present invention can be performed after a surgical lesion removal.

The ranges of numerical values indicated throughout the specification and claims include any value or any integer in between.

Each possibility disclosed throughout the specification represents a separate embodiment of the invention.

### Example 1

### Gel Formulation

The pharmaceutical formulation contained the following ingredients:

| | % (w/w) in | |
|---|---|---|
| Ingredient | formulation | Function |
| Proteolytic enzyme mixture | 5 | Active pharmaceutical |
| obtained from bromelain | | ingredient (API) |
| Guar gum | 3.5 | Gelling agent |
| Lactose | 16.05 | Anti-agglomeration agent |
| Potassium phosphate | 2.5 | pH adjusting agent |
| dibasic | | |
| Potassium phosphate | 0.8 | pH adjusting agent |
| monobasic | | |
| PEG-3350 | 1 | Anti-foaming agent |
| Water for injection | q.s. to 100%, | |
| | i.e., 71.15 | |

The pharmaceutical formulation was prepared by admixing the dried or powdered composition containing the proteolytic enzyme mixture (API), guar gum, lactose, potassium phosphate dibasic and monobasic, and PEG-3350, with water to form a hydrogel having a homogenous appearance and a viscosity ranging from 2,40,000 cP to 6,200,000 cP.

### Example 2

### Removal of Lesions by the Gel Formulation—Clinical Study

The aim of this study was to assess the safety and efficacy of the gel formulation of Example 1 on the removal of actinic (solar) skin lesions, specifically BCC, due to solar damage.

The subjects participated in this study were males or females, between 18 and 90 years of age, having a clinical suspicion of non-melanoma skin cancer or precancerous skin lesion, determined by medical history, physical examination, and dermatoscopy by a board of certified plastic surgeons and dermatologists. The subjects had skin lesions present for at least 4 weeks but no longer than 4 years. The skin lesions were characterized by a lesion surface area in the range of 0.5-2 cm². The skin lesions did not include mucosa and areas around the mouth, nostrils, eyes and ears.

Prior to application of the gel formulation, in some cases, the perimeter of few mm width around the lesion edges were protected with a thick layer of an adhesive barrier made of a paste, such as Hyaluronic acid gel, Petrolatum-based ointment, or Zink Oxide (ZnO₂), to protect the surrounding skin. The gel formulation was applied onto each lesion site for a time period of 9-12 hours, covered with occlusive film that adhered to the adhesive barrier, and the lesion site was covered with a bandage containing a non-absorbing dressing. Each lesion was photographed before and after each application.

This procedure was followed by up to 6 consecutive applications (i.e., 2-4 treatments per week), until a complete lesion destruction/removal was achieved. This period was denoted the “treatment period” (up to 3 weeks). The treatment period was followed by at least 4 weeks of “recovery period” which included treatment of the lesion site with a mild steroid ointment (e.g., Hycomycin 0.5%: hydrocortisone and neomycin; Betacorten G: gentamycin and betamethasone; 0.1%, or Aflumycin 0.16%: prednisolone and gentamycin), followed by a fatty ointment, applied once or twice daily until complete lesion healing was achieved.

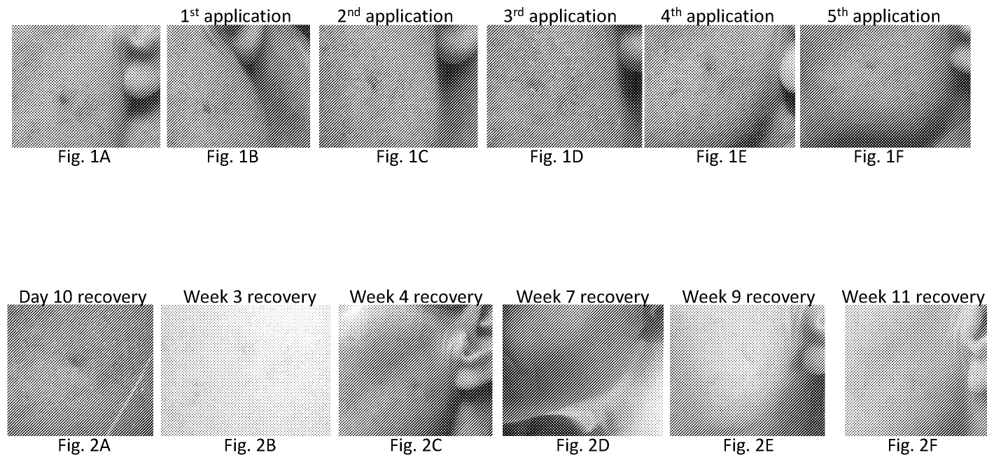

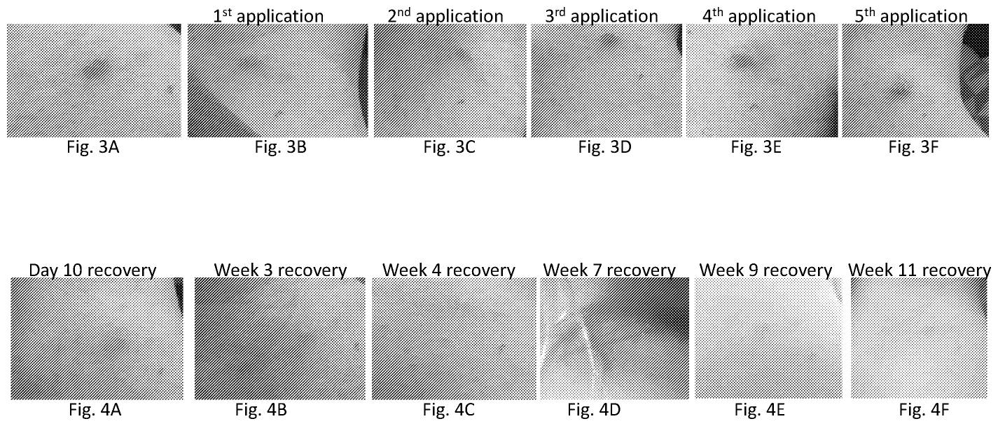

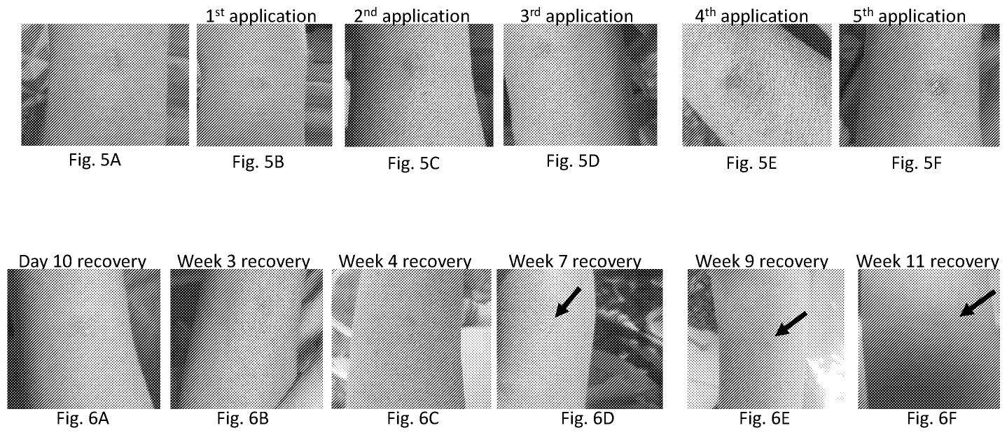

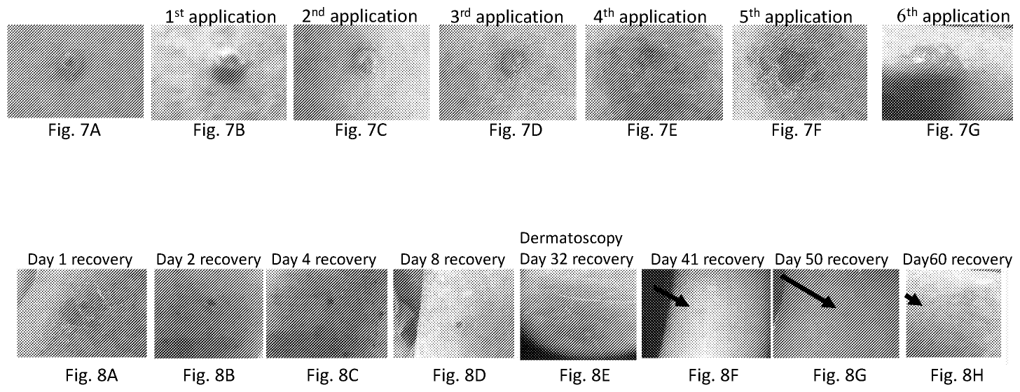

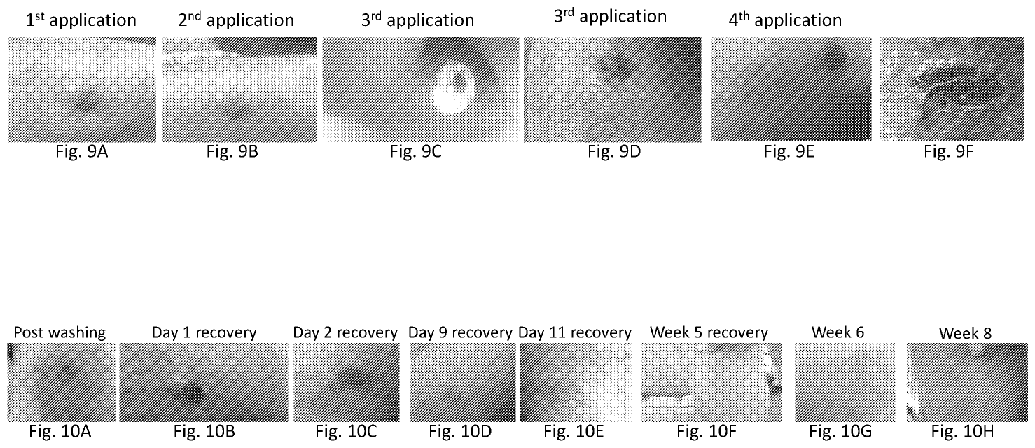

Representative photographs of the lesions before treatment, during the treatment period and during the recovery period are shown in FIGS. 1A-10H. As shown in the figures, all the BCC lesions were completely removed after 2-6 applications (“the treatment period”). Also, all the BCC lesions healed within less than 2 months, and in some cases in less than 4 weeks after the treatment period, when mild steroid and fatty ointments were applied to the lesion sites, with no detectable hypertrophic scarring (“the recovery period”, FIGS. 1-10). It was also observed that all the treated BCC lesion sites underwent complete regeneration and healing, and that the skin at the healed site maintained its integrity without any signs of tumor recurrence.

All patients complained of temporary skin irritation and mild itching pain that generally started around 1 hour after the first 1-3 applications and resolved untreated over the next 2-4 hours.

Mohs micrographic surgery (MMS) of the Morphea type BCC lesion and of the superficially invasive BCC lesion was performed and demonstrated that no residual cancer cells were present at the lesion sites. These results indicated that the destructive effect of the proteolytic enzyme mixture on residual cancer cells can have a long-term effect, i.e., delaying or preventing tumor recurrence.

### Example 3

### In Vitro Anti-Cancer Evaluation of the Proteolytic Enzyme Mixture Obtained from Bromelain

The aim of this study was to assess the anti-cancer or cytotoxic activity of the proteolytic enzyme mixture obtained from bromelain, also designated API, and to compare its activity to isolated stem bromelain, the major proteolytic enzyme of the API. Two cancer cell lines obtained from American Type Culture Collection (ATCC): basal cell carcinoma (BCC; TE 354.T; Human, CRL-7762) and squamous cell carcinoma (SCC; CAL-27; CRL-2095) were used to evaluate the cytotoxicity of the API or of stem bromelain as well as the growth inhibitory activity of these active agents on cancer cells. As control non-cancerous cells, human epidermal keratinocytes (HEK) were used.

All experiments were performed under sterile conditions.

Stem bromelain (200 mg) obtained from Sigma was dissolved in 1 ml double distilled water, filtered through a 0.2 m filter, aliquoted and stored at −20° C. At the day of the experiment, stem bromelain was diluted in cell culture media. The API was dissolved in water (activated) at the day of the experiment to reach the same stem bromelain content. The assay was carried out in triplicates. All cells were seeded in 96-well plates at a density of 150,00 cells/ml in 170 μl/well of fresh growth medium. The plates were incubated at 37° C. with 5% CO₂ until 70% confluence was reached. Thereafter, the medium was aspirated and replaced by fresh growth medium, i.e., DMEM, containing 1-200 μg/ml of either API or stem bromelain (170 μl/well).

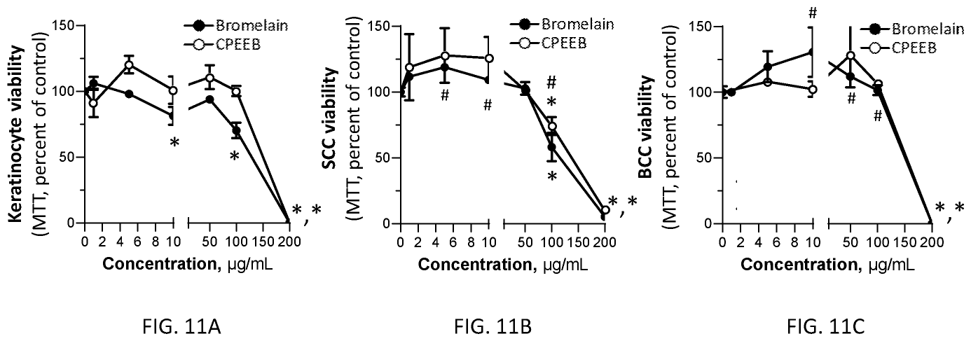

Naïve cells and cells treated with water in growth medium only served as negative controls. SDS (0.1%) was served as a positive control for viability. The cells were incubated at 37° C. with 5% CO₂ for 24 hours. At the end of the incubation time, the viability of the cells was measured using the MTT assay. As shown in FIG. 11A, after 24-hour incubation in the presence of 100 μg/ml of pure bromelain, HEK cell viability was reduced by 25%. However, incubation of HEK cells with the same concentration of the API (100 μg/ml) did not have any effect on HEK cell viability.

FIGS. 11B-C show that after 24 hours of incubation, no differences were observed between pure stem bromelain and the API in their effect on BCC and SCC cell line viability.

The results also indicate that while no significant effect on HEK cell viability was observed after 24-hour incubation in the presence of 100 μg/ml API, the viability of SCC cell line after the same incubation time and concentration of the API was reduced by 25.6%.

At 24-hour incubation, the vehicle had a negligible effect on cellular viability, while SDS totally abrogated cell viability.

These results therefore indicated that the cytotoxic effect of the API is more selective than that of pure stem bromelain as it affects SCC cell line viability but not HEK cell viability at a concentration of 100 μg/ml. The results further show that both the API and stem bromelain exert higher cytotoxic effect on SCC cell line than on BCC cell line under in vitro conditions.

The inhibitory effect of API on cell viability, apoptosis and cell proliferation was next examined.

The experiment was performed as described herein above with some modifications. Briefly, BCC, SCC and HEK cells were seeded in 96-well plates at a density of 150,00 cells/ml in 170 μl/well of fresh growth medium. The plates were incubated at 37° C. with 5% CO₂ until 70% confluence was reached. Thereafter, the medium was aspirated and replaced by fresh growth medium containing 50, 100, 150 and 200 μg/ml of either API or commercially available stem bromelain (170 μL/well). Naïve cells and cells treated with water in growth medium only served as negative controls. SDS (0.1%) was served as a positive control for viability, Staurosporine (10 μM) was used as a positive control for apoptosis and cell proliferation. The cells were incubated at 37° C. with 5% CO₂ for 30 hours. At the end of the incubation time, the viability of the cells was measured using the MTT assay, apoptosis was measured using Caspase-3 assay, and the turnover rate was measured by BrdU assay. The experiments were carried out in triplicates.

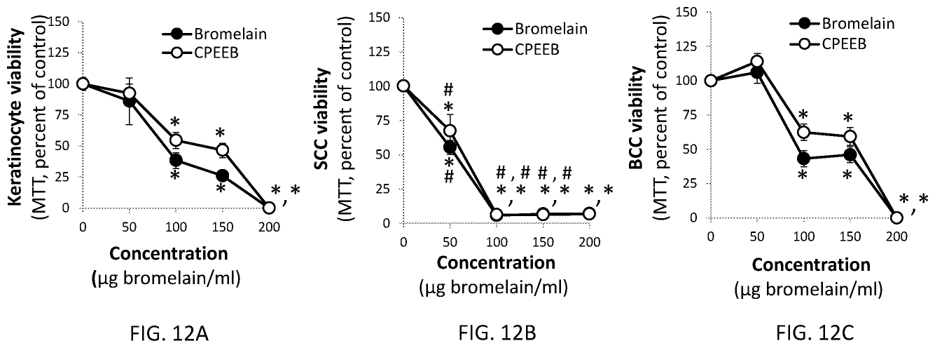

As shown in FIG. 12A, after 30 hours of incubation at concentration of 100 and 150 g/ml, stem bromelain exhibited higher cytotoxic effect on HEK cells than the API. At a concentration of 50 μg/ml of the API, HEK cell viability was not compromised (FIG. 12A), while at the same concentration of the API, SCC cell line viability was reduced to 67% (FIG. 12B). At 100 #g/ml of the API, HEK cell viability was 50% (FIG. 12A), while at the same concentration of the API, no viable SCC cells remained (FIG. 12B).

As shown in FIG. 12C, the cytotoxic effect of the API and of purified stem bromelain on the viability of BCC cell line was less pronounced than on SCC cell line. The LD₅₀ values of the API were 99.6, 100.9 and 49.9 μg/ml for HEK, BCC, and SCC, respectively. The LD₅₀ values of stem bromelain were 89.3, 124.6 and 50.12 μg/ml for BEK, BCC, and SCC, respectively. These results therefore indicate that SCC cells were significantly more affected by the API or stem bromelain treatment in comparison to normal BEK cells or BCC cells.

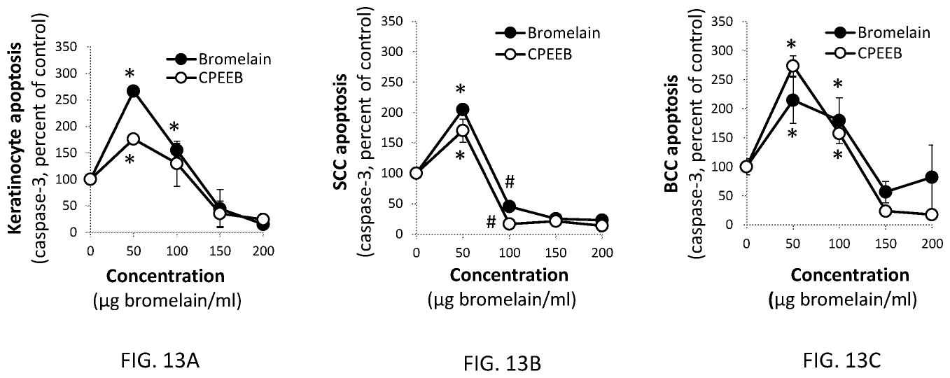

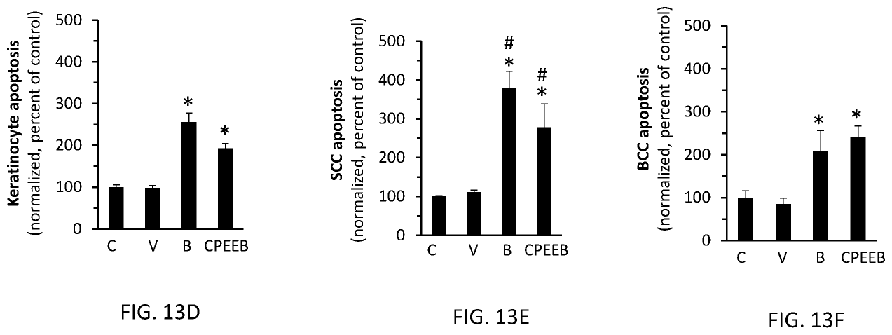

Cell death can be due to apoptosis or necrosis. In order to differentiate between these two processes, caspase 3 was used as biomarker for apoptosis. FIGS. 13A-F summarize the results of caspase expression. The results indicate that cell death (as observed by MTT assay) was correlated with the increase in Caspase-3 expression, indicating that cell death was due to apoptosis.

Protein expression is corelated with cell viability. To account for the reduction in cell number, caspase-3 activity assay at 50 μg/ml was normalized to the number of viable cells (as observed by the MTT assay). As can be seen in the FIGS. 13B, 13D and 13F, apoptosis was evident in all three cell lines. The expression level of caspase-3 after treatment with 50 g/ml of the API was significantly higher in SCC than in BCC or BEK cells. Staurosporine (used as positive control) enhanced significantly caspase-3 activity in all cells while the vehicle had no effect on caspase-3 expression.

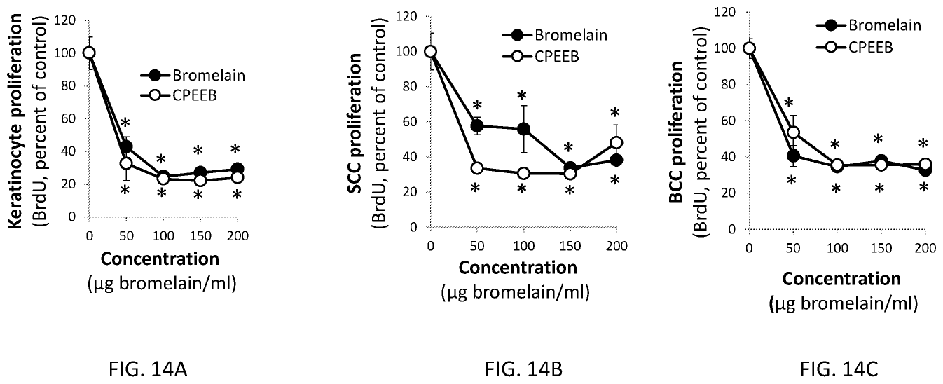

Cell proliferation was also investigated. The cells were exposed to BrdU at the last 24 hours of the 30-hour incubation period. The results indicated that cell proliferation was reduced by both the API and stem bromelain (FIGS. 14A-C). The proliferation of SCC cells treated with 50 and 100 μg/ml API was significantly lower than that of SCC cells treated with 50 and 100 μg/ml stem bromelain.

These results indicated that the cytotoxic effect of both the API and stem bromelain was selective, higher against SCC as compared to HEK or BCC cells. The cytotoxic effect of the API and stem bromelain was due to apoptosis. Both the API and stem bromelain reduced cell proliferation, but the inhibitory effect of API on SCC cell proliferation was more prominent than that of stem bromelain. In contrast, the cytotoxic effect of the API on normal cells was lower than that of stem bromelain. Thus, the API is found to be a more selective and hence a preferred anti-cancer agent than pure stem bromelain.

It will be appreciated by persons skilled in the art that the present invention is not limited by what has been particularly shown and described herein above. Rather the scope of the invention is defined by the claims that follow.

1.-35. (canceled)

36. A method of treating a skin lesion and delaying or preventing recurrence thereof comprising topically applying to a skin lesion of a subject in need of such treatment a therapeutically effective amount of a pharmaceutical formulation comprising:
(a) a composition in a dry or powdered form comprising:
(i) a proteolytic enzyme mixture obtained from bromelain comprising stem bromelain (EC 3.4.22.32);
(ii) a water-soluble gelling agent;
(iii) optionally, an anti-aggregation agent;
(iv) a pH adjusting agent; and
(b) water,
wherein, prior to use, the composition (a) is admixed with the water (b) to form the pharmaceutical formulation characterized by being a hydrogel having a pH ranging from about 6.0 to about 8.0,
wherein the amount of the proteolytic enzyme mixture obtained from bromelain ranges from about 0.5% (w/w) to about 10% (w/w) of the total weight of said pharmaceutical formulation, and
wherein the skin lesion is selected from the group consisting of a non-melanoma skin cancer, pre-cancerous skin lesions, and benign skin lesions, thereby treating said skin lesion and delaying or preventing recurrence thereof.

37. The method according to claim 36, wherein the non-melanoma skin cancer is selected from the group consisting of basal cell carcinoma (BCC), and squamous cell carcinoma (SCC).

38. The method according to claim 37, wherein the BCC is selected from the group consisting of superficial BCC, infiltrative BCC, nodular BCC, and combinations thereof.

39. The method according to claim 36, wherein the pre-cancerous skin lesion is selected from the group consisting of actinic keratoses, SCC in situ, a skin lesion due to Bowen's disease, actinic cheilitis, and moles.

40. The method according to claim 36, wherein the benign skin lesion is seborrheic keratosis.

41. The method according to claim 36, wherein the recurrence of the skin lesion is delayed for at least 18 to 24 months.

42. The method according to claim 36, wherein the pharmaceutical formulation is applied to the skin lesion for up to ten applications, or so long as said skin lesion is treated, and wherein said pharmaceutical formulation is maintained in contact with the skin lesion for at least about 4 hours up to 24 hours per application.

43. The method according to claim 42, wherein the pharmaceutical formulation is applied to the skin lesion for up to seven applications, and wherein said pharmaceutical formulation is maintained in contact with said skin lesion for about 8 hours to about 12 hours per application.

44. The method according to claim 42, wherein when the skin lesion is BCC, the pharmaceutical composition is applied to the skin lesion for five to seven applications, every other day, for about 9-12 hours per application.

45. The method according to claim 42, further comprising, following the up to ten topical applications of the pharmaceutical formulation, a step of topically applying to the skin lesion at least one healing composition selected from the group consisting of steroid ointments, fatty ointments, moisturizing creams, hydrophilic ointments, lotions, gels, sprays, aerosols, and combinations thereof.

46. The method according to claim 36, wherein the amount of the proteolytic enzyme mixture obtained from bromelain ranges from about 1% (w/w) to about 7% (w/w) of the total weight of the pharmaceutical formulation.

47. The method according to claim 36, wherein the water-soluble gelling agent is selected from the group consisting of naturally occurring gelling agents, semi-synthetic gelling agents, and synthetic gelling agents.

48. The method according to claim 47, wherein the water-soluble naturally occurring gelling agent is a polysaccharide selected from the group consisting of a galactomannan, a glucomannan, and a combination thereof.

49. The method according to claim 48, wherein the galactomannan is guar gum present in an amount ranging from about 0.25% (w/w) to about 5% (w/w) of the total weight of the pharmaceutical formulation.

50. The method according to claim 36, wherein the anti-aggregation agent is an oligosaccharide selected from the group consisting of lactose, sucrose, mannitol, and glucose.

51. The method according to claim 50, wherein the oligosaccharide is lactose present in an amount ranging from about 10% (w/w) to about 25% (w/w) of the total weight of the pharmaceutical formulation.

52. The method according to claim 36, wherein the pH adjusting agent is potassium phosphate present in an amount ranging from about 2% (w/w) to about 10% (w/w) of the total weight of the pharmaceutical formulation.

53. A method of treating a skin lesion and delaying or preventing recurrence thereof comprising topically applying to a skin lesion of a subject in need of such treatment a therapeutically effective amount of a pharmaceutical formulation comprising:
(a) a composition in a dry or powdered form comprising:
(i) a proteolytic enzyme mixture obtained from bromelain comprising stem bromelain (EC 3.4.22.32);
(ii) a water-soluble gelling agent of guar gum in an amount ranging from about 0.25% (w/w) to about 5% (w/w) of the total weight of the pharmaceutical formulation;
(iii) an anti-aggregation agent of lactose in an amount ranging from about 10% (w/w) to about 25% (w/w) of the total weight of the pharmaceutical formulation;
(iv) a pH adjusting agent; and
(b) water in an amount to complete to 100% (w/w) of the total weight of the pharmaceutical formulation,
wherein, prior to use, the composition (a) is admixed with the water (b) to form the pharmaceutical formulation characterized by being a hydrogel having a pH ranging from about 6.0 to about 8.0,
wherein the amount of the proteolytic enzyme mixture obtained from bromelain ranges from about 0.5% (w/w) to about 10% (w/w) of the total weight of said pharmaceutical formulation, and
wherein the skin lesion is selected from the group consisting of a non-melanoma skin cancer, pre-cancerous skin lesions, and benign skin lesions, thereby treating said skin lesion and delaying or preventing recurrence thereof.

54. The method according to claim 53, wherein the pharmaceutical formulation comprises:

| Ingredient | (%) w/w of formulation |
|---|---|
| Proteolytic enzyme mixture | 5 |
| obtained from bromelain | |
| Guar gum | 3.5 |
| Lactose | 16.05 |
| Potassium phosphate | 2.5 |
| dibasic | |
| Potassium phosphate | 0.8 |
| monobasic | |
| PEG-3350 | 1 |
| Water for injection | 71.15 |

55. A method of treating a skin lesion and delaying or preventing recurrence thereof comprising topically applying to a skin lesion of a subject in need of such treatment a therapeutically effective amount of a pharmaceutical formulation in the form of a hydrogel having a pH ranging from about 6.0 to about 8.0, the hydrogel formed from:
(c) a composition comprising:
(i) a proteolytic enzyme mixture obtained from bromelain comprising stem bromelain (EC 3.4.22.32);
(ii) a water-soluble gelling agent;
(iii) optionally, an anti-aggregation agent;
(iv) a pH adjusting agent; and
(d) water,
wherein the amount of the proteolytic enzyme mixture obtained from bromelain ranges from about 0.5% (w/w) to about 10% (w/w) of the total weight of said pharmaceutical formulation, and
wherein the skin lesion is selected from the group consisting of a non-melanoma skin cancer, pre-cancerous skin lesions, and benign skin lesions, thereby treating said skin lesion and delaying or preventing recurrence thereof.
